(19) United States
(12) Patent Application Publication
(10) Pub. No.: US 2019/0360005 A1
(43) Pub. Date: Nov. 28, 2019

(54) Method and Device for Producing Organic Compounds from Biogas

(71) Applicant: Siemens Aktiengesellschaft, München (DE)

(72) Inventors: Manfred Baldauf, Erlangen (DE); Elvira María Fernández Sanchis, Erlangen (DE); Marc Hanebuth, Nürnberg (DE); Katharina Meltzer, Erlangen (DE); Alexander Tremel, Möhrendorf (DE)

(73) Assignee: Siemens Aktiengesellschaft, München (DE)

(21) Appl. No.: 16/476,810

(22) PCT Filed: Jan. 10, 2018
(86) PCT No.: PCT/EP2018/050498

§ 371 Date: Jul. 9, 2019

(30) Foreign Application Priority Data
Jan. 12, 2017 (DE) 10 2017 200 435.5

(51) Int. Cl. C12P 7/06; C12M 1/107; C12P 7/16; C12P 7/18; C12P 7/30; C12P 7/54

(52) U.S. Cl. CPC C12P 7/065; C12M 21/04; C12P 7/16; C12M 43/00; C12P 7/30; C12P 7/54; C12P 7/18

(57) ABSTRACT

Various embodiments include a method for producing hydrocarbons, the method comprising: producing carbon monoxide and carbon dioxide with addition of oxygen in a first reaction unit; fermenting the carbon monoxide, the carbon dioxide, and hydrogen in a second reaction unit; adding biogas provided from a biogas system and oxygen from an electrolyzer as reactants to the first reaction unit; and adding hydrogen from the electrolyzer as a reactant to the second reaction unit.

### CROSS-REFERENCE TO RELATED APPLICATIONS

This application is a U.S. National Stage Application of International Application No. PCT/EP2018/050498 filed Jan. 10, 2018, which designates the United States of America, and claims priority to DE Application No. 10 2017 200 435.5 filed Jan. 12, 2017, the contents of which are hereby incorporated by reference in their entirety.

### TECHNICAL FIELD

The present disclosure relates to chemical processes. Various embodiments include systems and/or methods for producing organic compounds from biogas.

### BACKGROUND

Although there are currently numerous biogas systems in Germany, support for these systems under the Renewable Energy Sources Act is set to expire. These systems are typically used to operate block-type thermal power stations, which generate electrical energy and heat. After the above support expires, however, this will no longer be economical in all cases, and alternative use of the biogas system may therefore be desirable. Before it is fed into the German natural gas network, the biogas produced must first be processed by relatively costly methods. The treatment process is carried out in several steps: 1) removal of solid and liquid components and drying; 2) desulfurization; and 3) methane enrichment and separation of carbon dioxide. Biogas has a relatively high content of CO₂, most of which must be removed. In many cases, moreover, a calorific value adjustment is carried out using LNG, liquefied natural gas, which first is of fossil origin and second constitutes a cost factor.

### SUMMARY

The present disclosure describes further application possibilities for biogas systems other than those of the prior art. Some embodiments of the teachings herein may include a method for producing hydrocarbons (22), comprising the production of carbon monoxide and carbon dioxide with addition of oxygen (5) carried out in a first reaction unit (C1); fermentation with addition of the carbon monoxide produced, the carbon dioxide produced and hydrogen (4) carried out in a second reaction unit (C2); and use of biogas (12) provided by means of a biogas system (11) and oxygen (5) provided by means of an electrolyzer (3) as reactants for the first reaction unit (C1) and use of hydrogen (4) provided by means of the electrolyzer (3) as a reactant for the second reaction unit (C2).

In some embodiments, the reforming in the first reaction unit (C1) is carried out autothermically and in such a way that exit temperatures in the range of 550° C. to 1000° C., in particular between 580° C. and 850° C., are achieved.

In some embodiments, water (2) is supplied to the first reaction unit (C1) as a reactant, wherein a molar ratio of water (2) to oxygen (5) in the range of approx. 1.8 to approx. 3.8 is set.

In some embodiments, the first reaction unit (C1) comprises a catalyst containing Ni, Co, Zn, Cu and/or Mg, Ti, Pt and/or a rare earth element selected from cerium, yttrium or lanthanum.

In some embodiments, a portion of the biogas (12) is incinerated with the oxygen (5) in a combustion chamber (16) and the gas mixture thus produced is added together with the remaining biogas (12) and water (2) as reactants to the first reaction unit (C1).

In some embodiments, anaerobic gas fermentation, in particular by means of microorganisms of the genus Clostridium (C) such as e.g. C. ljungdahlii, C. autoethanogenum, C. ragsdalei, C. carboxidivorans, C. coskatti or the genus Moorella (M) such as e.g. M. thermoacetica, M. thermoautotrophica or Acetobacterium woodii or a coculture of one or a plurality of microorganisms, is carried out in the second reaction unit (C2).

In some embodiments, the produced hydrocarbons are ethanol, methanol, butyrate, formic acid, formiate, an acetyl complex, a coenzyme-A-activated acetate, acetone, butanol, hexanol, propanol, 2,3-butanediol or 1,3-propaneodiol.

In some embodiments, the reactant of the second reaction unit (C2) comprises less than 1000 ppmv of oxygen and less than 1 vol % of methane.

Some embodiments include a device for producing hydrocarbons, characterized by comprising the production of carbon monoxide and carbon dioxide with addition of oxygen (5) carried out in a first reaction unit (C1); the fermentation with addition of the carbon monoxide produced, the carbon dioxide produced and hydrogen (4) carried out in a second reaction unit (C2); and use of biogas (12) provided by means of a biogas system (11) and oxygen (5) provided by means of an electrolyzer (3) as reactants for the first reaction unit (C1) and use of hydrogen (4) provided by means of the electrolyzer (3) as a reactant for the second reaction unit (C2).

In some embodiments, the first reaction unit is an adiabatic fixed-bed reactor, a honeycomb reactor, a fluidized bed reactor or a tube bundle reactor.

### BRIEF DESCRIPTION OF THE DRAWINGS

The teachings are further described with reference to the figures. The figures show the following:

FIG. 1 shows a first example of a device incorporating teachings of the present disclosure;

FIG. 2 shows a second example of a device incorporating teachings of the present disclosure;

FIG. 3 shows a third example of a device incorporating teachings of the present disclosure;

FIG. 4 shows simulation results for selected operating points; and

FIG. 5 shows a schematic view of a method incorporating teachings of the present disclosure.

### DETAILED DESCRIPTION

In some embodiments, a method for producing hydrocarbons comprises the production of carbon monoxide and carbon dioxide with addition of oxygen carried out in a first reaction unit; fermentation with addition of the carbon monoxide produced, the carbon dioxide produced and hydrogen carried out in a second reaction unit; and use of biogas provided by means of a biogas system and oxygen provided by means of an electrolyzer as reactants for the first reaction unit and use of hydrogen provided by means of the electrolyzer as a reactant for the second reaction unit.

In some embodiments, a device for producing hydrocarbons comprises the production of carbon monoxide and carbon dioxide with addition of oxygen carried out in a first reaction unit; fermentation with addition of the carbon monoxide produced, the carbon dioxide produced and hydrogen carried out in a second reaction unit; use of biogas provided by means of a biogas system and oxygen provided by means of an electrolyzer as reactants for the first reaction unit and use of hydrogen provided by means of the electrolyzer as a reactant for the second reaction unit.

It should be noted that the term “hydrocarbon” is used here in the broad sense. This means that the target molecule comprises carbon and hydrogen, but can also comprise further elements such as e.g. oxygen and nitrogen. This term therefore also includes for example alcohols, ethers, or amino acids.

The chemical conversion of CO₂ into valuable products is currently a widely-discussed approach. For example, by means of the chemical reaction of CO₂ with H₂, the valuable products methanol and methane can be produced in a single step. However, the selection of products that can be efficiently produced by single-stage chemical synthesis is highly limited due to unfavorable equilibrium positions and low selectivity. However, direct production of more complex molecules, such as e.g. ethanol or butanol, is possible by means of biological fermentation, wherein gaseous CO₂ can also be used as a carbon source by means of so-called gas fermentation. In this process, CO₂ is converted by microorganisms specially selected for this purpose, such as e.g. anaerobic bacteria. As is the case in chemical synthesis, an energy-rich reaction partner is also necessary in order to allow conversion of CO₂. The required energy can also be provided by H₂, as is the case in chemical synthesis. The latter can be regeneratively produced using excess current or excess electric power by means of electrolysis. As an alternative to this, the bacteria can also use CO for energy production.

A special characteristic of gas fermentation of CO₂ and H₂ is that the absence of CO positively affects the selectivity and yield with respect to many target products, such as e.g. ethanol or butanol, and in many cases is necessary to make synthesis of the target products possible at all. However, CO is currently industrially obtained on a large scale mainly from fossil energy sources such as e.g. coal, natural gas, or petroleum. The methods and systems incorporating the teachings herein may include obtaining CO from regenerative biogas in a decentralized manner.

This approach is advantageous for example if the organic product of the gas fermentation is used for the production of propellants, as this product can then be counted as a biopropellant. In some embodiments, the methods and/or systems link an electrolyzer for the production of hydrogen and oxygen to a biogas system.

A gas fermentation system can be operated with a gas mixture of H₂, CO and CO₂, wherein the CO content can be obtained by reforming of biogas. The reforming can be carried out autothermically, i.e. without heating and without active cooling. The temperature required for reforming can be achieved by partial oxidation, which can be initiated by the addition of pure oxygen (O₂ content >90%). The reforming reactor can be operated in such a way that its exit temperature is in the range of 550° C. to 1000° C., in particular in the range of 580° C. to 850° C.

A portion of the hydrogen for gas fermentation can be derived from an electrolyzer in which water is decomposed. The oxygen produced can be fed into the reforming reactor. In some embodiments, at least 60% of the oxygen produced in electrolysis can be utilized, particularly advantageously at least 80%.

In addition to oxygen, water can also be converted in the reforming reactor. The molar ratio of water to oxygen can advantageously be in the range of 1.8 to 3.8. This ratio has a direct influence on the molar ratio of CO₂/CO for gas exiting the reforming reactor. The latter ratio is then in the range of 2 to 5.

The reforming reactor can comprise a catalyst that contains Ni, Co, Zn, Cu and/or Mg, Ti, Pt and/or a rare earth element, such as e.g. cerium, yttrium or lanthanum.

In some embodiments, the methods and/or systems include first incinerating a portion of the biogas with pure oxygen in a combustion chamber and then supplying the resulting gas mixture together with the remaining biogas and water to the reforming reactor in order to achieve a sufficiently high initial temperature for the reaction. In this case, the above-mentioned molar ratios refer to the reforming reactor and the combustion chamber as a whole.

The hydrogen produced in electrolysis can be fed together with the gas mixture produced in reforming to a gas fermentation system. This gas mixture can also contain hydrogen, wherein the total content of the hydrogen introduced into the second reaction unit can be between approx. 20% and approx. 80%.

In some embodiments, the gas fermentation carried out is anaerobic. In some embodiments, one can use the following microorganisms of the genus Clostridium (C) such as e.g. C. ljungdahlii, C. autoethanogenum, C. ragsdalei, C. carboxidivorans, C. coskatti or of the genus Moorella (M) such as e.g. M. thermoacetica, M. thermoautotrophica or Acetobacterium woodii or a coculture of one or a plurality of microorganisms.

In some embodiments, products of gas fermentation are in particular ethanol, methanol, butyrate, formic acid or a formiate, a complex of acetyl and coenzyme A “activated acetate”, acetone, butanol, hexanol, propanol, 2,3-butanediol, or 1,3-propanediol.

In some embodiments, the gas to be converted in the reforming reactor can be preheated with hot product gas from the reforming reactor via a heat exchanger. The gas fed into the gas fermentation system can comprise less than 1000 ppmv of O₂ and less than 1% CH₄. The reactor type can be an adiabatic fixed-bed reactor, a honeycomb reactor, a fluidized bed reactor or a tube bundle reactor.

In some embodiments, there is a gas reservoir for oxygen and hydrogen. This is not shown in the figures. In order to allow operation of the biogas system and electrolysis to be carried out at different times, this reservoir can hold oxygen and/or hydrogen. This makes it possible to continually operate the biogas system and the reforming reactor with approximately constant output without having to carry out electrolysis at the same time.

In some embodiments, an RWGS (reverse water-gas shift) reactor, a steam reformer, a dry reformer or a gasifier can be used for carrying out reforming in the first reaction unit.

In some embodiments, the electrolyzer is powered by means of regeneratively provided electrical energy, in particular surplus energy.

In some embodiments, at least 60% to 80% of the oxygen produced by means of the electrolyzer is utilized.

In some embodiments, gas derived from the first reaction unit comprises hydrogen, the content of which is adjusted to the total content of hydrogen brought into the second reaction unit in the range of approx. 20% to 80%.

In some embodiments, a heat exchanger, in particular a counterflow heat exchanger, is used to heat the reactant fed into the first reaction unit by means of the product gas of the first reaction unit.

In some embodiments, water in the gas mixture output from the first reaction unit is condensed out downstream of the heat exchanger and recycled to the first reaction unit or supplied to the electrolyzer.

In some embodiments, the first reaction unit is an adiabatic fixed-bed reactor, a honeycomb reactor, a fluidized bed reactor or a tube bundle reactor.

In some embodiments, a buffer reservoir can be used for the oxygen and hydrogen.

The gas mixture derived from the reforming reactor can comprise water. In some embodiments, the method and/or system may include condensing this water out downstream of the heat exchanger 14 and to recycle it into the process, specifically into the electrolysis or the reforming reactor. This is not shown in the figures.

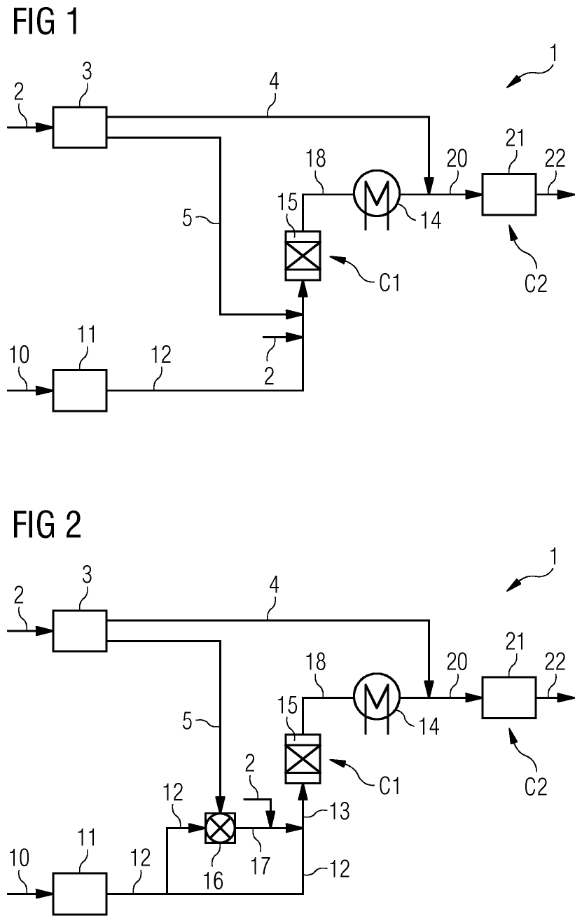

FIG. 1 shows a first example of a device 1 incorporating teachings of the present disclosure. In some embodiments, an electrolyzer 3 for the production of hydrogen 4 and oxygen 5 is linked to a biogas system 11. The biogas 12 comprises methane and a high content of CO₂. The methane is almost completely reacted in a reforming reactor C1, wherein for this purpose pure oxygen 5, specifically for partial oxidation, and water 2, specifically for steam reforming, are used. In some embodiments, the oxygen 5 is obtained from a water electrolyzer 3, and the hydrogen 4 produced in this process is mixed with the gas produced in reforming and fed to an anaerobically operated gas fermentation system C2. In some embodiments, pure oxygen 5, in particular instead of air, which is energetically more favorable overall, allows a greater amount of steam reforming can be carried out, which generates additional hydrogen 4 and results in smaller and thus more economical system components. In some embodiments, biogas systems 11 are often already equipped with desulfurization units, so that the biogas 12 can be used without problems in a reforming reactor C1. Simulation calculations have shown that the oxygen 5 and the hydrogen 4 from electrolysis can be almost completely utilized, a reforming reaction can be carried out autothermically, i.e. without an additional heat source or a cooling burden, and gas mixtures can be produced that show a suitable composition so that they can be directly used in anaerobic gas fermentation without requiring further addition of CO₂.

The anaerobic bacteria contained in the gas fermentation system C2 convert CO₂ as a carbon source and H₂ as an energy source and produce the target molecules. Moreover, they require CO for the production of many target molecules, wherein the requirement for CO is significantly lower than that for H₂. The device 1 provides that a portion of the required hydrogen 4 is obtained by means of an electrolyzer 3, and the CO is provided by reforming of biogas 12. In some embodiments, the CO₂ contained in the biogas 12 is utilized by means of dry reforming. The relevant reaction is as follows:

CH₄+CO₂→2 CO+2 H₂ ΔHᵣ⁰=247 kJ/mol (1)

The reaction provides a good possibility of effectively utilizing the high CO₂ content of the biogas 12. In this reaction, the two main components of the biogas, CO₂ and CH₄, react with each other and are thus consumed. This is absolutely necessary for the CH₄, as this compound cannot be used in the gas fermentation system C2. However, this reaction is not sufficient to convert all of the CH₄, as more CH₄ than CO₂ is present in the biogas 12 as a general rule. A possible way of avoiding this limitation is to add oxygen 5, which is derived in pure form directly from the provided water electrolyzer 3. In this manner, methane from the biogas 12 can be additionally converted by means of partial oxidation.

CH₄+1/2 O₂→CO+2 H₂ ΔHᵣ⁰=36 kJ/mol (2)

This reaction also has the advantage of being exothermic and thus allowing the reaction enthalpy required for dry reforming to be at least partially achieved. A further possible method of converting methane is steam reforming:

CH₄+H₂O_{(g)}→CO+3 H₂ ΔHᵣ⁰=206 kJ/mol (3)

Addition of water 2 can counteract carbonization, partly because the reaction temperature is reduced as this reaction is endothermic. At the same time, additional hydrogen 4 is produced, which can be used to advantage in the gas fermentation system C2. However, the addition of water 2 is disadvantageous in that the water-gas shift reaction can occur, thus further consuming CO.

CO+H₂O_{(g)}→CO₂+H₂ ΔHᵣ⁰=−41 kJ/mol (4)

Nevertheless, the reaction is exothermic, and thus in addition to the partial oxidation, helps to achieve the reaction enthalpy for the dry reforming and steam reforming. It is also advantageous that in addition to the CO, CO₂ is also required for the gas fermentation system C2.

The device 1 is characterized in that a biogas 12, if necessary after desulfurization, is converted on a catalyst. The aim is for any methane present to be completely converted, with makes addition of oxygen 5 and/or water 2 necessary. An autothermic reaction process C1 is considered to be particularly advantageous, as this process requires no additional heat source and also does not require cooling. The system then aims for thermodynamic equilibrium, wherein a specified composition and a specified temperature at the reactor outlet have been achieved when equilibrium is reached.

In FIGS. 1 to 3, the following reference numbers have the following meanings. Reference number 1 represents a device incorporating teachings of the present disclosure. Reference number 2 denotes supplied water. Reference number 3 denotes an electrolyzer by means of which H₂ and O₂ are produced. Reference number 4 denotes hydrogen. Reference number 5 denotes oxygen. Reference number 10 denotes the addition of biomass. Reference number 11 denotes a biogas system. Reference number 12 denotes biogas. Reference number 13 denotes biogas that is reacted with oxygen, water and any combustion products present originating from a combustion chamber 16. Reference number 14 denotes a heat exchanger. Reference number 15 denotes a reforming reactor. Reference number 17 denotes a hot gas from a combustion chamber 16. Reference number 18 denotes a gas mixture of CO₂, CO, H₂ and H₂O. Reference number 20 denotes the supply of gas to the gas fermentation system C2. Reference number 21 denotes a gas fermentation system C2, which can consist of a plurality of fermenters. Reference number 22 denotes an organic valuable product as a hydrocarbon to be produced. FIG. 2 shows a second example of a device 1 incorporating the teachings of the present disclosure. Here, the reference numbers of FIG. 1 correspond to those of FIG. 2. The new reference number 16 denotes a combustion chamber. It can be advantageous first to incinerate a portion of the biogas 12 with pure oxygen 5 in the combustion chamber 16 and then to feed the resulting gas mixture together with the remaining biogas 12 and water 2 into the reforming reactor C1 in order to achieve a sufficiently high initial temperature for the reaction. In this case, the above-mentioned molar ratios of water 2 to oxygen 5 and carbon dioxide to carbon monoxide refer to the reforming reactor and the combustion chamber as a whole.

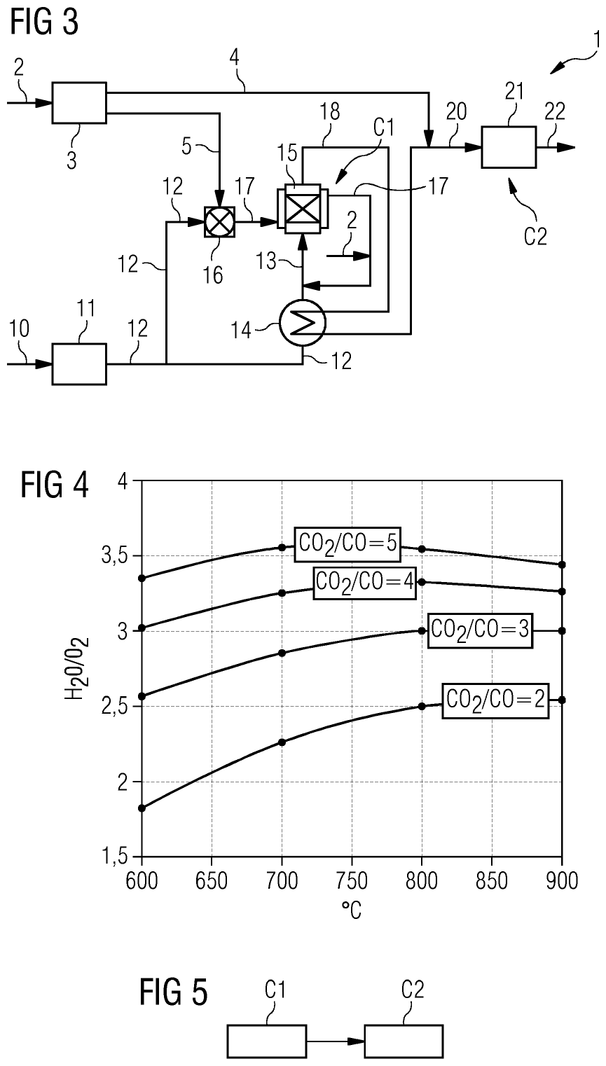

FIG. 3 shows a third example of a device 1 incorporating teachings of the present disclosure. The invention provides a way of retrofitting existing biogas systems 11 such that they can be used to produce higher-value organic target products 22. For this purpose, the biogas system 11 is combined with a reforming reactor C1 and an electrolyzer 3 that produces hydrogen 4 using regenerative energy. The gas mixture is fed into a gas fermentation system C2 that is also novel, in which the actual target product 22 is produced. Compared to the alternative technological route of “biomass gasification/gas purification/adjustment of syngas composition by means of CO shift/CO₂ separation,” the approach presented here offers the use of existing systems, specifically a biogas system 11 including any gas purification if present, to which one can resort, wherein these systems could be given “a second life” after expiration of the EEEG incentive for biogas systems. In this manner, investments already made could be safeguarded, and it would not be necessary to make investments in new systems for the alternative utilization of biomass via gasification.

In some embodiments, existing biogas systems 11, which do not require a special substrate such as e.g. glucose, are already equipped with a desulfurization unit, so that the biogas 12 can either be directly introduced into a reforming reactor C1, or only fine purification is required before said introduction. In this combination, the fact that electrolysis produces pure oxygen 5 in addition to hydrogen 4 may provide a major advantage. By means of partial combustion of the biogas 12, the reforming C1, which is predominantly highly endothermal dry reforming, can be carried out in a highly efficient manner, wherein carbon formation on the catalyst is simultaneously counteracted.

CO₂ produced is converted in the gas fermentation system C2. In simulation calculations, operating states were identified in which the gas fermentation system C2 requires no addition of further CO₂. In some embodiments, moreover, water 2 is converted in the reforming reactor, so that hydrogen 4 is produced by means of additional steam reforming, which reduces the requirement for hydrogen from electrolysis. Calculations show that this reduction can be from 20% to 80%, wherein the actual numerical value depends on the composition of the biogas 12 and the desired gas composition for the gas fermentation system C2. Furthermore, the use of pure oxygen 5 means that no nitrogen gets into the process. No nitrogen oxides are produced in the reforming reactor C1, and the system components are smaller overall, which makes the entire process more economical. In addition, the absence of nitrogen reduces the energy required for compressing the feed gases before they enter the fermenter C2.

FIG. 4 shows a diagram of simulation results for selected operating points. In a process according to FIG. 1, a biogas 12 with a specified composition has two variable parameters: mass flow of added O₂ and mass flow of added H₂O. By skillfully selecting these two parameters, a specified reaction temperature, and thus a desired ratio of CO₂ to CO at the reactor outlet, can be set. Depending on the application in question, molar ratios of CO₂ to CO of 2 to 4 are required for a gas fermentation system C2. Efforts are currently being made to further reduce the content of CO so that ratios of 5 will also appear to be suitable in future approaches. Using a model biogas composed of 60% CH₄ and 40% CO₂, simulation calculations were carried out in order to determine whether these required molar ratios can be set by means of an equilibrium conversion and what amounts of oxygen and water must be added for this purpose. At the same time, the aim was to ensure that no significant amounts of CH₄ and O₂ remain in the gas mixture for gas fermentation, i.e. that these two gases are almost completely converted. These conditions are met for the points shown in FIG. 4.

FIG. 4 shows simulation results for selected operating points in the autothermic conversion of a gas having a composition of 60% CH₄ and 40% CO₂ by adding O₂ and H₂O to thermodynamic equilibrium. At the points shown, there are no significant amounts of CH₄ or O₂ in the gas produced. By varying the molar ratio of the H₂O and O₂ (y axis) added, specified CO₂/CO ratios in the product gas can be set in a defined manner. Higher temperatures can generally be achieved by adding a larger amount of oxygen. This is not shown in FIG. 4.

The simulation results show that a suitable temperature window for the reaction lies in the range of 550° C. to 850° C. At lower temperatures, significant amounts of CH₄ remain present in the gas mixture, which, however, cannot be utilized in gas fermentation. At higher temperatures, the amount of hydrogen formed is reduced, so that an electrolyzer would have to be larger to achieve the same production capacity of a gas fermentation system. This is therefore the optimum temperature range from an economic standpoint for the operation of a system incorporating the teachings herein. In some embodiments, a very high proportion of the oxygen produced in electrolysis, and possibly all of it, can be utilized, which also leads to more economical operation. The result is a gas which, after addition of the hydrogen 4 from the electrolysis, has the exact composition required for anaerobic gas fermentation C2, with no further addition of CO₂ being necessary.

FIG. 5 shows a schematic view of the method incorporating the teachings herein. C1 constitutes a step of reforming and C2 a step of gas fermentation.

What is claimed is:

1. A method for producing hydrocarbons, the method comprising:
producing carbon monoxide and carbon dioxide with addition of oxygen in a first reaction unit;
fermenting the carbon monoxide, the carbon dioxide, and hydrogen in a second reaction unit;
adding biogas provided from a biogas system and oxygen from an electrolyzer as reactants to the first reaction unit; and
adding hydrogen from the electrolyzer as a reactant to the second reaction unit.

2. The method as claimed in claim 1, wherein the process in the first reaction unit is carried out autothermically with exit temperatures in the range of 550° C. to 1000° C.

3. The method as claimed in claim 1, further comprising adding water to the first reaction unit as a reactant;
wherein a molar ratio of water to oxygen is in the range of approx. 1.8 to approx. 3.8.

4. The method as claimed in claim 1, wherein the first reaction unit comprises a catalyst containing at least one element selected from the group consisting of: Ni, Co, Zn, Cu, Mg, Ti, Pt, cerium, yttrium, and lanthanum.

5. The method as claimed in claim 1, further comprising incinerating a portion of the biogas with the oxygen in a combustion chamber; and
adding a resulting gas mixture together with remaining biogas and water as reactants to the first reaction unit.

6. The method as claimed in claim 1, further comprising anaerobic gas fermentation in the second reaction unit with microorganisms of the genus Clostridium (C) and/or a coculture of one or a plurality of microorganisms.

7. The method as claimed in claim 1, wherein the produced hydrocarbons comprise at least one compound selected from the group consisting of: ethanol, methanol, butyrate, formic acid, formiate, an acetyl complex, a coenzyme-A-activated acetate, acetone, butanol, hexanol, propanol, 2,3-butanediol, and 1,3-propaneodiol.

8. The method as claimed in claim 1, wherein the reactant of the second reaction unit comprises less than 1000 ppmv of oxygen and less than 1 vol % of methane.

9. A device for producing hydrocarbons, the device comprising:
a first reaction unit for the production of carbon monoxide and carbon dioxide with addition of oxygen;
a second reaction unit for the fermentation of the carbon monoxide produced, the carbon dioxide produced, and additional hydrogen; and
using biogas provided from a biogas system and oxygen provided from an electrolyzer as reactants for the first reaction unit; and
using hydrogen provided from the electrolyzer as a reactant for the second reaction unit.

10. The device as claimed in claim 9, wherein the first reaction unit comprises at least one of: an adiabatic fixed-bed reactor, a honeycomb reactor, a fluidized bed reactor, and a tube bundle reactor.
